(19) United States
(12) Patent Application Publication
(10) Pub. No.: US 2006/0079895 A1
(43) Pub. Date: Apr. 13, 2006

(54) Methods and devices for improved bonding of devices to bone

(76) Inventor: Thomas J. McLeer, Redmond, WA (US)

Correspondence Address: WILSON SONSINI GOODRICH & ROSATI, 650 PAGE MILL ROAD, PALO ALTO, CA, 94304-1050, US

(21) Appl. No.: 11/140,570

(22) Filed: May 26, 2005

Related U.S. Application Data
(60) Provisional application No. 60/614,712, filed on Sep. 30, 2004.

(51) Int. Cl. A61F 2/30

(52) U.S. Cl. 606/61

(57) ABSTRACT

The present invention is directed to improving bonding between orthopedic devices, particularly vertebral devices, and bone. The present invention provides various methods and devices employing mechanical and bio-fixation modalities for such attachment. As provided herein, the initial mechanical attachment of a device to bone is sufficiently stable to ensure that the implanted device is relatively immobile (or alternatively microscopic motion is promoted), facilitating bone and soft tissue in-growth and the eventual bio-fixation of the device.

### PRIORITY CLAIM

This patent application claims the benefit of previously-filed U.S. Provisional Pat. No. 60/614,712, filed Sep. 20, 2004, and entitled “Novel Anchor Fixation to the Pedicle.”

### FIELD OF THE INVENTION

The present invention relates generally to the field of surgical implants and orthopedics, and in particular to novel methods and devices for improved anchoring, and/or bonding, of orthopedic devices to bone.

### BACKGROUND OF THE INVENTION

Fixation and repair devices for the treatment of various orthopedic injuries and diseases are well known in the art and include devices such as plates, pins, screws, anchors, rods, joint replacements and the like. These devices typically are made of biocompatible materials including metallic alloys, composite materials, memory alloys, ceramics and/or carbon fiber materials. Depending upon the objectives of the orthopedic procedure, the associated devices can (1) provide temporary support, and/or securement, of anatomical structures until natural healing mechanisms can repair damaged tissues (with the healed tissues eventually bearing some or all of the natural anatomical loads); or (2) can be designed to provide long-term support, in conjunction with, or in place of, damaged or destroyed tissues. Where long-term support is needed or desired, these devices may comprise materials that generally do not corrode, or otherwise degrade, inside a patient's body. Shorter term support, on the other hand, can involve materials that: degrade, and/or dissolve, over time; that are incorporated or absorbed by the body; or that are designed to be removed eventually from the body.

In either case, successful implantation and performance of fixation devices often hinges on their ability to adhere, and maintain, permanent attachment to bone and/or other anatomical structures. It is difficult to achieve direct bonding between bone and orthopedic devices, especially on a long-term, load-bearing basis, where immediate fixation strength is also desired (such as when immediate ambulation and/or load-bearing by the bone and/or surrounding tissues is desired). One method, however, is to mechanically “lock” the implant to the surrounding bone using screw threads and/or locking pins, i.e., intermedullary rods with cross-locking screws, pedicle screws, etc. However, when such an implant is subjected to cyclic loading, various repetitive stress-related failures can often occur, including: (1) implant failure; (2) bond/interface failure; and (3) bone failure.

In addition to mechanically securing orthopedic devices to bone, adequate fixation of the device may be ensured through the use of cements or other types of adhesives. Despite this, migration and/or loosening of these devices after implantation is not uncommon. Points of failure may include the interface between the bone and cement/adhesive or the integrity of the cement/adhesive and/or the bone itself. Failure is often due to the various stresses and strains that operate to weaken the bonds within the bone and within the device and adhesive, as well as the adhesive itself. Although methods have been developed to improve the properties of bone cements and adhesives, the inherent limitations of these materials are increasingly apparent and other techniques for improving device fixation are needed.

### SUMMARY OF THE INVENTION

It has been suggested that natural bone and/or soft tissue in-growth into, on, and/or around implanted devices might provide a clinically acceptable alternative to the use of cements and adhesives. This biological in-growth may serve as an alternative, or supplemental, technique to other attachment modalities, and can provide enhanced interfacial strength between bone and orthopedic devices, sufficient to support load bearing devices, as well as overcome some of the drawbacks of using cement or adhesives. Further, because osteoclasts and osteoblasts desirably remodel damaged bone over time, microscopic damage and/or fractures induced and/or caused by repetitive loading of the bone and/or implant can be repaired. In order to exploit biological in-growth as a means for device attachment, the device will desirably be secured in a stable position, generally with little or no significant movement, while it is in intimate contact with the bone.

The present invention is directed at providing stable mechanical attachment of various fixation devices to bone in order to allow immediate and/or less-delayed loading of the implant following implantation while concurrently promoting bone and soft tissue in-growth for device attachment over long periods. These, as well as other advantages of the present invention, are detailed herein.

The present invention is further directed to bonding various orthopedic devices to bone, and in particular, vertebral prosthesis and vertebral fixation devices. The present invention provides methods and devices employing both immediate and long term fixation modalities (in one example, mechanical and biological) for attachment and load bearing. In accordance with various embodiments of the present invention, the mechanical attachment of a device to bone is desirably and sufficiently stable to ensure that the device remains relatively immobile relative to the surrounding bone, providing immediate stability and support (desirably promoting intimate contact between the device and surrounding tissues) while facilitating long-term bio-fixation. “Bio-fixation,” as used herein, refers to an attachment modality wherein a device is secured to bone via soft-tissue, and/or bone in-growth into, on or around a device, supplementing and/or replacing mechanical fixation or attachment. In various embodiments, bio-fixation may occur relatively quickly, such as within a few minutes or hours, or over longer time periods, such as weeks or months. Bio-fixation, as used herein, can encompass various attachment methodologies (or combinations thereof) such as natural healing reactions (including, but not limited to, calcification, osteophytic bone growth or scarification), chemically or biologically enhanced healing reactions (utilizing osteoinductive or osteoconductive substances) or varying types of biologically-induced mechanical fixation (adhesion).

In yet another aspect of the invention, a method for securing a device to bone comprises the use of a device having at least one mechanical fixation region, and at least one bio-fixation region, wherein the at least one mechanical fixation region is sized and configured to securely attach the device to bone and to maintain the integrity of device fixation during normal physiological loaded and/or unloaded conditions, while desirably facilitating long-term fixation of the bio-fixation to bone. In one embodiment, the mechanical fixation of the device prevents significant movement of the device, promoting bio-fixation such as biological in-growth. In an alternate embodiment, microscopic motion of the device after implantation is permitted and/or even desired in order to promote or accelerate the bio-fixation, and/or reduce stresses experienced by the implant and/or bone.

In another embodiment, a method for securing a device to bone comprises: attaching mechanically at least a portion of the device to the bone so as to provide an initial attachment of the device to the bone to permit some load-bearing; and promoting biological in-growth to facilitate the subsequent bio-fixation of the device.

In another aspect of the present invention, a device having at least one mechanical fixation region, and at least one bio-fixation region, is provided; wherein the mechanical fixation region is configured to be securable to bone in order to provide stable mechanical attachment, facilitating subsequent bio-fixation.

In another aspect of the invention; a device has at least one mechanical fixation region which also incorporates one or more bio-fixation elements in the same region. For example, such a device could incorporate screw threads having a cutting surface that incorporates one or more bio-active, or bio-fixable, materials within the threads, between the threads, within the grooves and/or incorporated onto or into the shaft of the screw. Similarly, the device could incorporate openings or voids that are empty upon implantation, or filled with bioactive substances that break down and create voids over time for bone in-growth. Similarly, the device could comprise mechanical fixation regions formed from bio-fixation substances.

In a further aspect of the present invention, the mechanical fixation region may comprise one or more engagement mechanisms. Examples of these mechanisms include, but are not limited to, any type of threaded engagement mechanism (such as those used in conventional screw fixation devices), clamping or engaging mechanisms (teeth, jaws, compression clamps, etc.) and compression/expansion mechanisms (such as wedging and/or expanding anchors). In other examples, the mechanical fixation region comprises one or more engagement mechanisms and elements, wherein the elements are adapted to prevent rotation and migration of devices during bio-fixation. These elements include, but are not limited to, various wings, blades, paddles, helical and longitudinal projections, rods, resorbable rods and the like as described in: “Anti-Rotation Fixation Element for Vertebral Prostheses,” by Leonard J. Tokish et al., Ser. No. 10/831,657 filed Apr. 22, 2004 (which is herein incorporated by reference in its entirety); and as is further described below. In other examples, one or more conventional engagement mechanisms can be combined with one or more elements adapted to prevent migration and/or rotation of the device within or from the bone.

In one embodiment, a portion of the device comprises a fixation anchor, or “sleeve,” incorporating bio-fixation elements, delivered in a percutaneous and/or minimally-invasive fashion into the targeted bone region. Desirably, the anchor will bond with the surrounding bone over a period of days, weeks or months, and once sufficient bonding has occurred, the remainder of the device can be mechanically attached to the anchor. In various embodiments, the “sleeve” could comprise device(s) that can be safely and effectively delivered to a treatment site in a patient while under local anesthetic, preferably in an out-patient procedure.

In other examples, the mechanical fixation region can further comprise bone cement and/or other adhesives to enhance the mechanical attachment of the device at the fixation region. However, as described below, bone cement and other adhesives tend to inhibit biological in-growth, and their use is desirably limited to the mechanical fixation regions of the device. In a preferred embodiment, the bone cement will not encroach into the bio-fixation regions, and will remain a sufficient distance away from these regions (as well as the vascular regions which supply them with nutrients) to allow for sufficient bio-fixation to occur. In a similar manner, the resorption of various biological cements (calcium phosphate, hydroxy-apatite, etc.), which is often resorbed (and new bone laid down) by the action of osteoclasts/osteoblasts, can be significantly affected by the presence of bone cement/other adhesive components, and thus should be isolated from such materials, if possible.

The bio-fixation region of the device is adapted to promote and/or accelerate bone and soft tissue in-growth, further securing the device to bone. In some examples, the bio-fixation region comprises one or more of the following biocompatible materials, including, but not limited to: osteoconductive, osteoinductive and/or bone scaffolding materials; bone graft materials; biologically resorbing cements; biologically active coatings incorporating bone modifying proteins (BMPs) or other growth peptides.

In other examples, one or more surfaces of a device within one or more regions can be adapted to promote biological in-growth for attachment of the device. These adaptations include, but are not limited to: chemical etching; grit blasting; and various porous coating techniques (Tecotex®, sintered coatings, etc.) to promote bone and soft tissue in-growth.

In various embodiments, the mechanical fixation region(s) can be separated to some degree (or “isolated” to varying degrees) from the biological fixation area(s). Depending upon the type and/or quantity of mechanical fixation desired, as well as the type and/or quantity of biological fixation desired, the method of mechanical fixation may adversely affect the biological fixation area's ability to bio-fixate to the surrounding anatomy. Similarly, the bio-fixation type can adversely affect the ability of the mechanical fixation region to adequately secure the implant initially and/or over the length of time necessary for adequate bio-fixation to occur. For example, in the case of mechanical fixation using bone cement, and bio-fixation using a bony in-growth surface, the monomer used in the bone cement can inhibit and or destroy the actions of the osteoclasts and/or osteoblasts responsible for bone growth into the bony in-growth structures. By separating the mechanical and bio-fixation areas, the monomer will desirably be isolated from the bio-fixation areas. Alternatively, the bio-fixation region could incorporate a bio-degradable “sealant” or additive that prevents the monomer from entering the bio-fixation region while the bone cement is curing and subsequently break down after the monomer (or other component or components having adverse effects on bone remodeling) has dissipated.

These and other embodiments and features are described in further detail in the following description related in the appended drawings.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1a is an exploded perspective view depicting various components of a facet replacement prosthesis, which includes a fixation member and an artificial facet joint structure, both of which are connected by a system of connections;

FIG. 1b is a cut-away top plan view of the fixation member implanted into the pedicles of a targeted vertebral body;

FIG. 1c is a cut-away top plan view of an alternate embodiment of a fixation member implanted into the pedicles of a targeted vertebral body;

FIG. 1d is a cut-away top plan view of another alternate embodiment of a fixation member implanted into the pedicles of a targeted vertebral body;

FIGS. 1e through 1g are cut-away top plan views of another alternate embodiment of a fixation member implanted into the pedicles of a targeted vertebral body;

FIG. 2a is a perspective view of a device comprising one or more blades on a proximal section of the device to resist rotational and/or lateral forces upon device implantation;

FIG. 2b is a cross-sectional view of the device of FIG. 2a, taken along line 2b-2b;

FIG. 3 is a perspective view of a device comprising one embodiment of a paddle for resisting rotational and/or lateral forces upon device implantation;

FIG. 4 is a perspective view of a device illustrating yet another embodiment of a paddle;

FIG. 5 is a perspective view of a device having a bent fixation member comprising helical longitudinal depressions;

FIG. 6a is a perspective view of an alternate embodiment of a fixation member constructed in accordance with the teachings of the present invention;

FIG. 6b is a transverse cross-section view of the embodiment of FIG. 6a taken along lines 6b-6b;

FIG. 7a depicts one embodiment of a mechanical locking device suitable for use with the various embodiments disclosed herein; and

FIG. 7b depicts an alternate embodiment of a mechanical locking device suitable for use with the various embodiments disclosed herein.

### DETAILED DESCRIPTION OF THE PREFERRED EMBODIMENTS

Although the present disclosure provides details enabling those skilled in the art to practice the various embodiments of the invention, it should be understood that the physical embodiments provided herein merely exemplify the invention, which may be embodied in other specific structures. Accordingly, while preferred embodiments of the invention are described, details of the preferred embodiments may be altered without departing from the invention. All embodiments that fall within the meaning and scope of the appended claims and equivalents thereto are therefore intended to be embraced by the claims.

The features of the present invention may be used or incorporated, with advantage, on a wide variety of medical devices, and in particular with the vertebral systems, including but not limited to, conventional vertebral fixation devices as well as those facet replacement, or arthroplasty, systems and devices specifically described in: “Facet Arthroplasty Devices And Methods”, by Mark A. Reiley, Ser. No. 09/693,272, filed Oct. 20, 2000, now U.S. Pat. No. 6,610,091, issued Aug. 26, 2003; “Prostheses, Tools And Methods For Replacement Of Natural Facet Joints With Artificial Facet Joint”, by Lawrence Jones et al., Ser. No. 10/438,295, filed May 14, 2003; “Prostheses, Tools And Methods for Replacement Of Natural Facet Joints With Artificial Facet Joint”, by Lawrence Jones et al., Ser. No. 10/438,294, filed May 14, 2003; “Prostheses, Tools And Methods For Replacement Of Natural Facet Joints With Artificial Facet Joint”, by Lawrence Jones et al., Ser. No. 10/615,417, filed Jul. 8, 2003; “Polyaxial Adjustment Of Facet Joint Prostheses”, by Mark A. Reiley et al., Ser. No. 10/737,705, filed Dec. 15, 2003; and Anti-Rotation Fixation Element for Vertebral Prosthesis”, by Tokish, et al., Ser. No. 10/831,657 filed Apr. 22, 2004; all of which are hereby incorporated by reference for all purposes. It should be noted that while the embodiments of the present invention are described with respect to facet arthroplasty systems, the present invention can be used in conjunction with other vertebral systems and devices as well as other prosthesis systems for the treatment of non-vertebral diseases and injuries, including but not limited to, the treatment of hips, knees, arms, shoulders, wrists and the like.

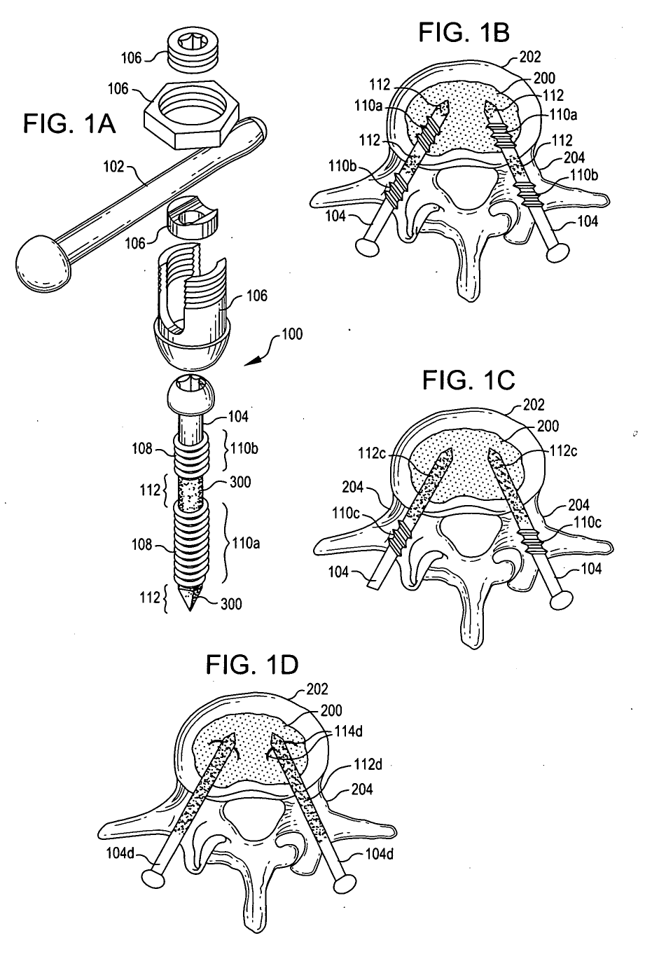

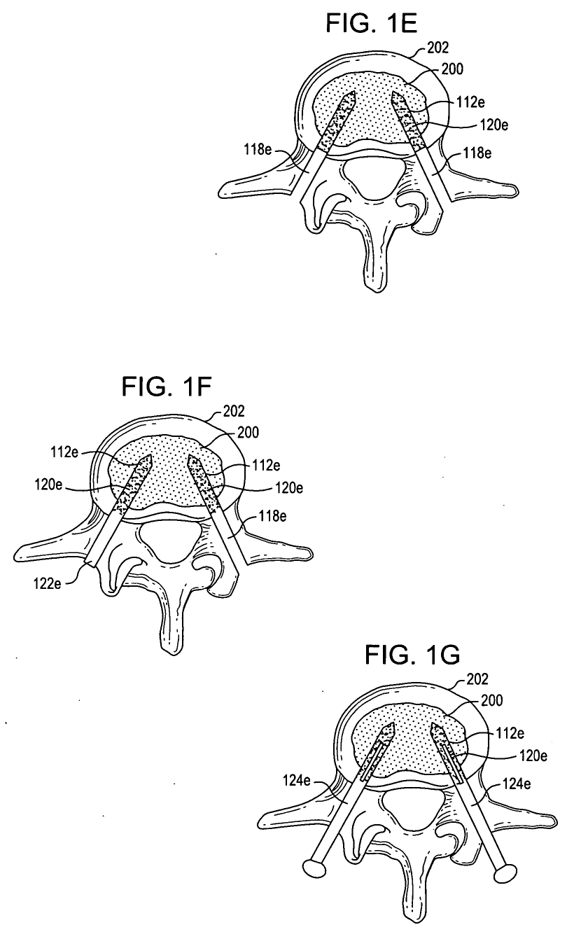

Turing now to the drawings, FIG. 1a illustrates one embodiment of a vertebral prosthesis 100 employing features of the present invention. In this example, the prosthesis 100 is an artificial facet joint prosthesis, specifically an artificial cephalad facet joint prosthesis, which can be used to replace the inferior portion of a natural facet joint, as further described in Reiley et al., Ser. No. 10/737,705, the disclosure of which is incorporated herein by reference. The prosthesis 100 is implantable directly into a vertebra and configured to articulate with other components of the facet prosthesis system, such as those described in Reiley, et al., Ser. No. 10/737,705. The prosthesis 100 desirably mates and functions in conjunction with the superior half of a facet joint, which may be a natural facet joint or yet another artificial facet joint prosthesis, such as a caudal facet joint prosthesis. One or both inferior facet joints on a single vertebra can be replaced using prosthesis 100 as described in Reiley et al., Ser. No. 10/737,705.

As pictured in FIG. 1a, the vertebral prosthesis 100 comprises various components, including an artificial facet joint structure 102, which is coupled to a fixation element 104 via a system of connections 106, which permits the facet joint structure 102 and the fixation element 104 to rotate and/or move with respect to each other relative to one or more axis. The prosthesis 100 is secured into the bone via implantation of the fixation element 104 into the vertebral body via or at the pedicles and/or lamina. As illustrated, the series of threads 108 located in the mechanical fixation regions 110 serve to stably attach the prosthesis 100 into the bone. It should be noted that while the fixation element 104 is described generally as a screw, specifically a pedicle screw comprising threads 108 in mechanical fixation regions 110, other fastening and joining mechanisms can be employed. Examples of these mechanisms include, but are not limited to: the use of stems, rods, anchors, clips, cables and the like, all of which are within the scope of the present invention. In addition, thread geometries as well as the pitch of threads 108 can be adapted to further enhance threaded fixation of the prosthesis 100 into bone. Preferably, the initial mechanical attachment of the prosthesis 100 is secure and stable so that there is no significant movement of fixation element 104, relative to the surrounding bone structure, to promote bone and soft tissue in-growth within the bio-fixation regions 112.

In the embodiment shown in FIG. 1b, a first mechanical fixation region 110a can be desirably positioned within a cancellous bone region 200 of the vertebral body 202, and a second mechanical fixation region 110b can be desirably positioned within the pedicle 204 of the vertebral body 202. Because the pedicle 204 comprises a relatively thicker shell of strong cortical bone, the positioning of the mechanical fixation region 110a within, and in intimate contact with, this surrounding cortical bone structure desirably allows for significant strength of mechanical fixation, while concurrently allowing biological fixation to occur within, and adjacent to, the bio-fixation regions 112.

FIG. 1c depicts an alternate embodiment of a fixation element in which fixation element 104c incorporates a single mechanical attachment region 110c and at least one extended bio-fixation region 112c. In this embodiment, the position, type and orientation of the mechanical fixation region is desirably chosen to correspond to a region of the targeted bone that is best suited for immediate strong mechanical fixation (in this example, the interior of the pedicle 204), while maximizing the remaining surface area of the fixation element 104 available for biological fixation (in this example, biological fixation may occur within the cancellous bone as well as within a portion of the cortical bone of the pedicle).

In various other embodiments, the mechanical and bio-fixation regions may be specifically designed or adapted to take advantage of the surrounding anatomy, including the location and quality of cancellous bone, cortical bone, muscles, cartilage and connective tissues. For example, the structural properties of cancellous bone (en masse) are not isotropic—i.e.: cancellous bone's ability to withstand load is often dependent upon the orientation of the load. In the case of the vertebral body, the structural properties of the cancellous bone are generally transversely isotropic (i.e. cancellous bone in the vertebral body generally withstands medial-lateral or anterior/posterior loading to a different extent than cephalad-caudal loading). Accordingly, an anchor specifically designed to maximize the transverse surface area and/or reduce the cephalad-caudal surface area could be similar in design to the fixation element or anchor depicted in the embodiment of FIG. 3.

FIG. 1d depicts another alternative embodiment of a fixation element 104d constructed in accordance with the teachings of the present invention, in which the fixation element 104d incorporates one or more distally-located mechanical locking struts 114d and at least one bio-fixation region 112d. In this embodiment, the locking struts 114d, which may comprise memory metal such as Nitinol, etc., extend into the surrounding cancellous bone region 200 of the vertebral body 202 when the fixation element 104 is in a desired position within the bone. Desirably, the struts 114d will mechanically secure the fixation element 104d in its desired position until the bio-fixation region 112d is biologically anchored to the bone. If desired, mechanical fixation within the pedicle can be further augmented using screw threads within the pedicle as well.

FIGS. 1e through 1g depict another alternative embodiment of a fixation element 104e constructed in accordance with the teachings of the present invention, in which the fixation element 104e incorporates a distally positioned anchor 120e having a bio-fixation outer surface 112e. Desirably, a physician can create one or more channels 118e in a targeted bone using preferably minimally-invasive techniques (as depicted in FIG. 1e), in order to implant one or more anchors 120e into the patient's bone. Desirably, biological fixation secures the anchors 120e in position over time, while the one or more removable plugs 122e (as depicted in FIG. 1f) occupying the remaining portions of the channel 118e and are not fixed to the bone. Desirably, the plugs 122e will occupy various region(s) of the implant, thereby preventing soft/hard tissue from occupying growing into areas of the implant designated for ultimate fixation to support bodies 124e. Once the anchor 120e has been sufficiently fixated to the bone (which can potentially be analyzed using radio-graphic imaging, through MRI or CTI scanning, or the like), the plugs 122e can be removed during a full surgical procedure, and support bodies 124e (as depicted in FIG. 1g) can be inserted into the channel 118e and mechanically anchored to the anchors 120e (using screw threads, etc), thereby immediately accomplishing a biologically fixated construct immediately adapted to withstand loading.

The various bio-fixation regions desirably comprise material or materials 300 that promote and/or accelerate bone and tissue in-growth within these areas so that the eventual bio-fixation of the prosthesis to bone is facilitated. The bio-fixation regions can comprise, but are not limited to, one or more of the following: osteoconductive, osteoinductive and/or bone scaffolding materials; bone graft materials; biologically active coatings incorporating bone modifying proteins (BMPs) or other growth peptides. Alternatively, the bio-fixation regions could comprise chemically etched surfaces, roughened surfaces, porous coatings, grit blasted surfaces and/or similarly textured surfaces to promote biofixation and bio-ingrowth within these regions. If desired, the bio-fixation material can be formed integrally with the device, or the bio-agents can be added to the device at the time of the surgical procedure(s). In alternative embodiments, the bio-agents could be stored or contained within a resorbable membrane that will resorb/dissolve after implantation. Material choice considerations can include one or more of the following: physician preference, patient needs and/or anatomical suitability to various forms and types of bio-agent.

In various embodiments, bone cement and/or an adhesive can be applied to the various mechanical fixation regions to enhance the mechanical attachment of the fixation element(s) into the vertebra. Where some bone cement(s) and/or adhesive(s) tend to inhibit bone and soft tissue in-growth, the use of these materials would desirably be limited to the mechanical fixation regions and the migration of such substances (or their biological effects) into the bio-fixation regions would be inhibited and/or prevented. Accordingly, in various embodiments, one or more gaps may be formed or left between the mechanical and bio-fixation regions, or one or more cement restrictors or flow restrictors can be placed between these various regions. In addition or alternatively, bioactive/bio-degradable sealants can be used to inhibit cement or adhesive flow into the bio-fixation region(s). In the case of a sealant (including materials that can be used as sealants such as Poly Lactic Acid, Poly Glycolic Acid or calcium sulfate, etc.), the sealant or other like material could comprise a bio-active, bio-degradable or hydrolytic-degradable material which desirably prevents bio-inhibitive materials from migrating into the bio-fixation region(s), but which eventually allows bio-in growth to occur there-through (for example, the sealant could degrade within the human body, thereby allowing subsequent infusion of biogrowth therethrough). In alternative embodiments, resorbable/remodelable bioactive cements (such as calcium phosphate or Norian® Skeletal Repair Cement) could be incorporated around and/or in the implanted device, or manufactured as part of the cement or other securement component of the implanted device.

As another alternative, the mechanical and bio-fixation regions could comprise a single securement region of a similar construction (such as a uniform porous coating, etc.) with the adhesive material (or mechanical interlock with the surrounding anatomy) securing some sections of the securement region and bio-fixation securing others.

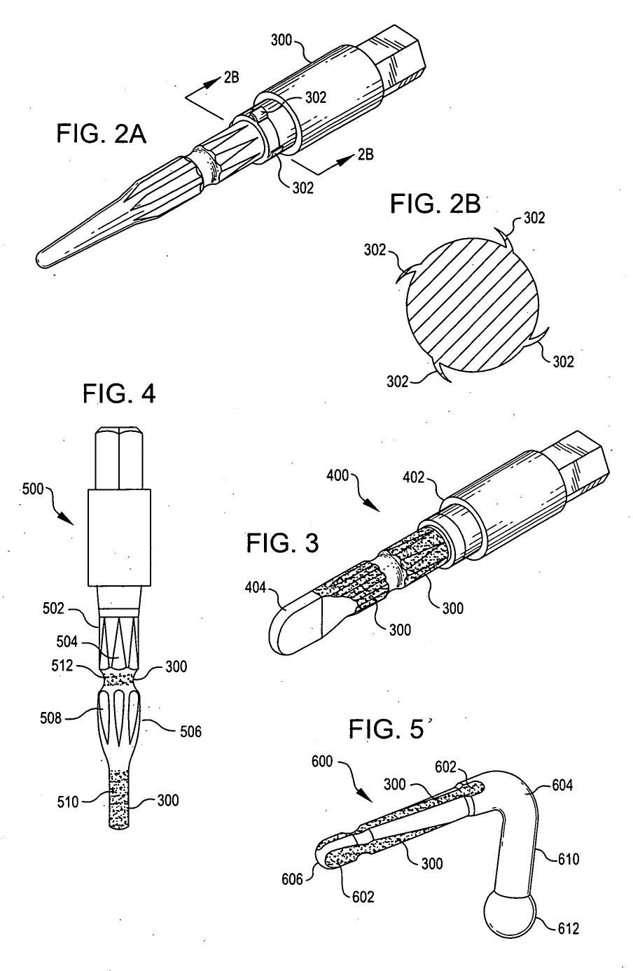

FIGS. 2-6b depict various other alternative embodiments incorporating alternative mechanical engagement mechanisms and/or elements to provide enhanced fixation into bone. Generally, these engagement elements are adapted to overcome or withstand rotational and/or lateral forces (torsional and/or axial forces, respectively) typically imparted on orthopedic devices upon implantation into bone. More detailed descriptions and other embodiments of various engagement elements (or “anti-rotation” or “anti-pull” members) are provided in “Anti-Rotational Fixation Element for Vertebral Prostheses,” Ser. No. 10/831,657. It should be understood, however, that one or more of the elements described therein can be incorporated into or combined with any of the embodiments of the present invention despite the fact that not all the members and features discussed therein are expressly illustrated in the preferred embodiments of the present invention.

In the alternative embodiment of FIGS. 2 and 2b, the mechanical fixation region incorporates one or more directional fins or spikes 302 which desirably permit rotation in one direction but inhibit rotation in the opposing direction. Spikes 302 comprise a rigid, semi-rigid or flexible material (or some combination thereof, including some or all of the material comprising memory metal such as Nitinol, etc.) that is secured at one end to fixation member 300 and which extends outward of the surface on fixation member 300. Desirably, spike 302 is biased-shaped to present a relatively smooth surface to surrounding tissue when rotation in one direction (in the example of FIG. 2b, this direction would be clockwise rotation out-of-the-page), but which presents a sharp or flattened surface to surrounding tissue when rotated in the opposite direction. Where spikes 302 are relatively non-rigid, rotation of the anchor in one direction would desirably tend to compress the spikes against the surface of the anchor, allowing relatively free rotation, while reverse rotation of the fixation member 300 would induce the spikes 302 to dig into the surrounding tissue, thereby inhibiting rotation in that direction.

FIG. 3 depicts another alternative embodiment of a fixation element constructed in accordance with various teachings of the present invention. In this embodiment, the fixation element 400 comprises an elongated body 402 having a flattened tip 404 at the distal end. As previously noted, flattened tip 404 will desirably present an increased surface area to relatively weaker areas of surrounding bone (not shown), thereby reducing the force per unit area experienced under loading conditions experienced by the surrounding bone. In this embodiment, bio-fixation materials 300 can be incorporated into the shaft 300 at various locations, including one or more positions between the body 402 and flattened tip 404, as well as along the face of the flattened tip 404, if desired.

FIG. 4 depicts another alternative embodiment of a fixation element 500 constructed in accordance with the various teachings of the present invention. In this embodiment, fixation element 500 incorporates an anti-pull out feature. As used herein, an anti-pull out feature refer to an element or combination of elements which acts to mitigate, minimize or counteract forces bearing upon the prosthesis portion or fastener to disengage, loosen, pull or otherwise axially translate the fastener relative to the vertebra. The fixation element 500 shown in this figure includes a proximal grooved portion 502 having proximal grooves 504 and a distal grooved portion 506 having distal grooves 508. Proximal grooves 504 have a proximal tip with a width that increases distally and distal grooves 508 have a nearly constant width terminating in a distal tip 510. A reduced diameter portion 512 separates the proximal grooved portion 502 from the distal grooved portion 506. The proximal grooves 504, distal grooves 508 and reduced diameter section 512 act to increase the surface area of the vertebral fixation element 500. By increasing the surface area of the vertebral fixation element 500, this embodiment provides greater attachment between this device 500 and the vertebra. The greater amount of surface area may be used advantageously with material or materials 300 that promote and/or accelerate bone and tissue in-growth within these areas so that the eventual bio-fixation of the prosthesis to bone is facilitated. The greater surface area allows more material or materials 300 to be present along the length and a particularly greater amount of such material to be present about the reduced diameter section 512. The increased amount of material or materials 300 present adjacent the reduced diameter portion 512 produces a section of increased diameter that counteracts pull out forces.

Next, FIG. 5 illustrates an embodiment of a vertebral prosthesis fixation element 600 with helical longitudinal depressions 602 as anti-rotation elements and a fixation element with a bend 604. The illustrated embodiment of the vertebral prosthesis portion 600 has a distal tip 606 and a proximal end 610. The proximal end 610 includes a socket element 612 for further attachment or interaction to another vertebral prosthesis. The plurality of longitudinal depressions 602 extending from the distal tip 606 to the proximal end 610 increase the surface area of vertebral prosthesis fixation element 600. The increased surface area allows for more area to support biofixation materials thereon. It is to be appreciated that the longitudinal depressions 602 may also be varied. It is to be appreciated that each of the longitudinal depressions 602 has a longitudinally varying profile, narrowing as the longitudinal depression extends proximally. In alternative embodiments, the longitudinally varying profile can widen or remain constant as the longitudinal depression extends proximally. Although in the illustrated embodiment all of the longitudinal depressions are identical, in other embodiments, the multiple longitudinal depressions can differ, for example by having different profiles, lengths, starting and/or ending points, etc. Alternative embodiments can have one longitudinal depression, two longitudinal depressions, four longitudinal depressions, five longitudinal depressions, or more longitudinal depressions. If desired, the distal tip 606 of the device can incorporate a helical or corkscrew-type extension (not shown) to further engage the surrounding bone.

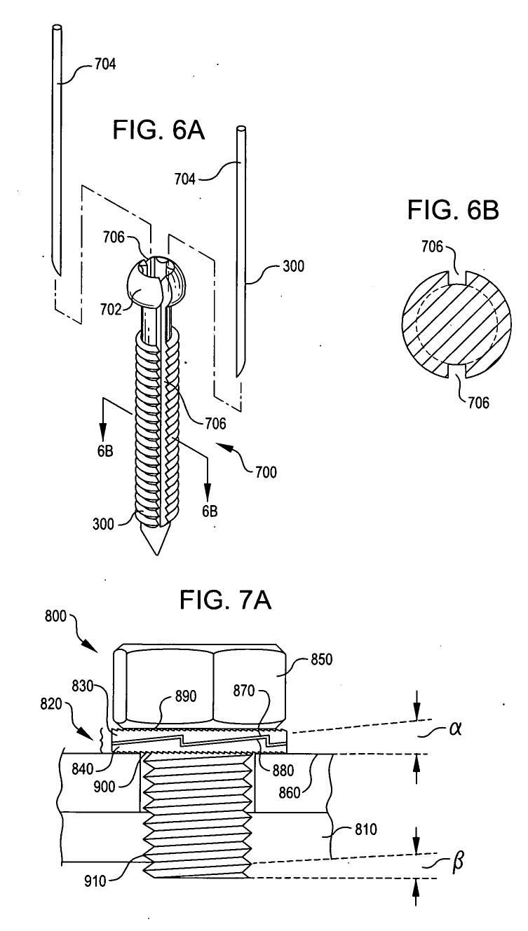

FIGS. 6 and 6b depict another alternative embodiment of a fixation element 700 constructed in accordance with various teachings of the present invention. In this embodiment, the fixation element 700 comprises an interrupted-screw anchor 702 and one or more pins 704. Formed along on or more sides of anchor 702 are one or more slots or channels 706 sized and configured to accept the pins 704 therein: In use, the anchor 702 can be threaded into the targeted bone in a known manner. Once in a desired position, pin 704 can be advanced down the slot 706, desirably locking the anchor 702 in position and inhibiting and/or preventing subsequent rotation of the anchor 702. If desired, pin 704 and/or anchor 702 can comprise a bio-fixation material 300 which provides for eventual bio-fixation of the anchor/pin to the surrounding anatomy. If desired, the anchor may be “capped” (not shown) after insertion of the pin(s) to ensure that the pins do not subsequently migrate and/or dislodge by sliding towards and past the head of the anchor 702.

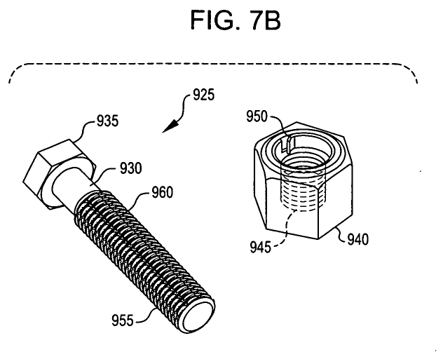

FIGS. 7a and 7b depict alternate embodiments of self-locking devices useful in conjunction with the teachings and embodiments of the present invention. In FIG. 7a, a bolt 800 is secured to a member 810. A split washer 820 having a first portion 830 and a second portion 840 is positioned between the head 850 of the bolt and an outer surface 860 of the member 810. The first and second portions 830 and 840 each have respective inner faces 870 and 880 and outer faces 890 and 900. In this embodiment, the bolt incorporates a right-handed securing thread 910 having a securing thread pitch β, and the inner faces 870 and 880 of the split washer 820 each have a cooperating locking bevel angle α.

In this embodiment, the securing thread pitch β is desirably less than the locking bevel angle α, such that, if the bolt attempts to rotate counterclockwise (such as in an attempt to self-loosen, for example), this rotation of the bolt will desirably cause a commensurate rotation of the first portion 830 of the split washer (desirably, the bolt and split washer are interlocked in some manner such that they rotate concurrently). Because the bevel angle of the split washer is greater than the pitch of the thread, counterclockwise rotation of the bolt will desirably cause the split washer to separate to a greater degree than the equal amount of rotation withdraws the screw threads from the member 810. In this manner, the counterclockwise rotation will actually tighten the resulting bond between the bolt and the member 810. Desirably, the outer face 900 of the second portion 840 will incorporates a surface having both a mechanical locking element (such as teeth, for example) and a biological locking element (such as a bony in-growth surface, for example) to permit both immediate and long-term fixation of the bolt. In an alternative embodiment, other portions of the bolt, including the screw threads, the head, or portions of the split washer, can incorporate biological fixation elements.

FIG. 7b depicts another alternative embodiment of a self-locking device 925 which incorporates a mechanical locking-mechanism which desirably prevents (or reduces the opportunity for) inadvertent loosening of the device from surrounding hard tissue. In this embodiment, the locking mechanism is designed to allow for immediate mechanical fixation with surrounding hard tissue while concurrently facilitating biological fixation between the device and the surrounding tissue.

Self-locking device 925 comprises a bolt 930 having a head 935, and a nut 940 having an interior threaded section 945 and a locking detent 950. The bolt 930 further has a series of screw threads 955, with each screw thread 955 incorporating a series of notches 960 which cooperate with the locking detent 950 of the nut 940 to permit the bolt 930 to be tightened onto the nut 940, but which inhibits loosening of the bolt 930.

In use, the bolt 930 can extend through a targeted member (such as a targeted bone or other hard tissue—not shown), with the nut 940 threaded onto and tightened on the distal end 960 of the bolt 930 which extends out of the member, with the member being compressed between the head 935 of the bolt 930 and the nut 940. Alternatively, a nut-shaped recess could be formed into the member (using a chisel or punch, for example), the nut positioned within the recess, and the bolt could be threaded through the nut 940 and then into the member, with the screw threads holding the bolt 930 within the member, and the notches 960 interacting with the detent 950 to prevent removal and/or loosening of the bolt from the member.

If desired, various bone-contacting surfaces, such as the outer surface of the nut 940, or the side surfaces of the nut and head, or the various surfaces of the bolt, could incorporate biological fixation surfaces, such as bony in-growth surfaces, in accordance with the various teachings of the present invention. In a similar manner, the components described in the various disclosed embodiments, and their equivalents, could incorporate varying degrees of mechanical and/or biological fixation, with varying results.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for securing a device to bone comprising: using a fixation device having at least a first fixation region and at least a second fixation region-wherein the first fixation region is adapted for initial mechanical attachment of the device to bone for facilitating biological ingrowth into the second fixation region.

2. The method of claim 1, wherein the mechanical attachment of the device to bone is sufficient to provide initial load bearing functionality until subsequent bio-fixation of the device is established.

3. The method of claim 1 wherein the second fixation region is physically separated from the first fixation region.

4. The method of claim 3 wherein mechanical fixation of the device prevents significant movement of the device.

5. The method of claim 3 wherein the first fixation region is adapted to facilitate microscopic movement of the device to promote bio-fixation.

6. The method of claim 4 wherein the first fixation region of the device is adapted to prevent pull-out of the device after implantation thereof into bone.

7. The method of claim 6 wherein the first fixation region of the device is adapted to prevent rotation of the device after implantation thereof into bone.

8. The method of claim 1 wherein the second fixation region of the device comprises a surface adapted to promote bio-fixation of the device into bone.

9. The method of claim 3 wherein the second fixation region of the device comprises a material for promoting bio-fixation of the device into bone.

10. The method of claim 1 wherein the first fixation region is isolated from the second fixation region.

11. The method of claim 10 wherein the first fixation region comprises bone cement.

12. The method of claim 11 wherein the first fixation region and second fixation region are isolated by a structure configured to prevent migration of the bone cement into the second fixation region.

13. The method of claim 1 wherein the second fixation region comprises one or more mechanical fixation structures.

14. The method of claim A13 wherein the mechanical fixation structure is a strut.

15. An orthopedic device comprising: a first attachment region having one or more mechanical structures that are adapted to securely attach said device to bone; and a second attachment region which is adapted to facilitate bio-fixation of the device.

16. A method of implanting a fixation device into a patient's vertebra to promote bio-fixation of said device comprising: implanting the device having an elongated body wherein a portion of the elongated body is positioned within a cancellous bone region of the vertebra and a second portion of the elongated body is positioned within a cortical bone region.

17. The method of claim 16 further comprising implanting the device through a pedicle.

18. The method of claim 17 wherein the second region is adapted to ensure mechanical attachment of the device into the vertebra and the first region is adapted to promote bio-fixation of the device.

19. The method of claim 18 wherein the mechanical attachment provides sufficient load-bearing support to prevent significant displacement of the device.

20. The method of claim 19 wherein the second portion promotes bio-fixation of the device.

21. The method of claim 20 wherein the second portion of the device comprises one or more mechanical fixation structures.

22. A method of attaching an orthopedic device into bone, said method comprising:
implanting an anchoring device into bone;
promoting bio-fixation of the anchoring device to provide sufficient load bearing support; and
coupling the anchoring device to the orthopedic device.

23. The method of claim 22 wherein a channel is created in the bone to facilitate implantation of the anchoring device into the bone.

24. The method of claim 23 wherein a surface of the anchoring device is adapted to promote bio-fixation of the anchoring device within the bone.

25. The method of claim 24 wherein the orthopedic device is directly attached to the anchoring device.

26. The method of claim 22 wherein the anchoring device is implanted into a cancellous bone region of a patient's vertebra.
